(19) United States
(12) Patent Application Publication
(10) Pub. No.: US 2024/0175854 A1
(43) Pub. Date: May 30, 2024

(54) AIR POLLUTION FORECAST MANAGEMENT SYSTEM AND AIR POLLUTION FORECAST MANAGEMENT METHOD

(71) Applicant: FINETEK CO., LTD., NEW TAIPEI CITY (TW)

(72) Inventors: Chao-Kai CHENG, NEW TAIPEI CITY (TW); Szu-Wei HUANG, NEW TAIPEI CITY (TW); Yu-Wen CHEN, NEW TAIPEI CITY (TW); Chung-Hsiang MU, NEW TAIPEI CITY (TW)

(21) Appl. No.: 18/108,029

(22) Filed: Feb. 10, 2023

(30) Foreign Application Priority Data
Nov. 30, 2022 (TW) 111145874

(51) Int. Cl. G01N 33/00; G01W 1/10; G16Y 20/10; G16Y 40/10

(52) U.S. Cl. CPC G01N 33/0063; G01N 33/0034; G01N 33/0067; G01N 33/0075; G01W 1/10; G16Y 20/10; G16Y 40/10

(57) ABSTRACT

An air pollution forecast management system including an air quality management device and an Internet of Things (IOT) cloud platform is disclosed. The air quality management device includes a dust particle sensing module being configured to sense gas exhausted from a smoke exhaust flue. The IoT cloud platform is configured to compute, at a second time after a first time, an exhaust gas set of the gas drifting from the first time to the second time by using current-observed meteorological data at the second time, receive a plurality of air-pollution sets at a plurality of geographic locations at the second time, compute a plurality of influencing results of the plurality of air-pollution sets respectively associated with the exhaust gas set, and generate a feedback instruction according to at least one of the plurality of influencing results to control gas emission of the smoke exhaust flue.

### BACKGROUND OF THE DISCLOSURE

### Technical Field

The disclosure generally relates to a management system and a management method, and more particularly to a management system and a management method for forecasting gas tracks of the gas exhausted by factories and providing feedback information accordingly.

### Description of Related Art

The factories exhaust gas or waste air during production processes, and the gas or the waste air includes particulate matter and dust. Generally, the particulate matter and dust drift in the air and then settle on the ground. The finer the particle is, such as PM2.5 (2.5 μm) and PM10 (10 μm), the longer time in the air the particle drifts.

The factories increase industry gas exhaustion amount for economic activity, so air pollution also increases. When the factories constantly exhaust the gas, air pollution cumulates in some particular areas, so human beings and creatures feel uncomfortable and the gas may make them unhealthy.

The particles of the gas exhausted by the factories diffuse with different moving paths, speeds, and directions resulting from the meteorological condition (temperature, humidity, wind direction, wind speed) where the factories locate, and the influence on the surrounding environment changes over time. The related art monitors the amount of gas exhausted from the smoke exhaust flue of the factory and prompts some warnings accordingly: however, the warnings are not directed to the influence caused by the factories to the surrounding environment. Even if the gas exhaustion amount of the factories is decreased according to the instant warning, the air pollution has cumulated in the air and makes a negative influence on the surrounding environment because other pollution sources (such as smog or the gas exhausted by other factories nearby) also affects the air quality of the surrounding environment, and it is too late to decrease the amount of the gas exhaustion amount at the moment. There is no effective management strategy currently for the factories in exhausting the gasfactory.

There is a trade-off problem between economic activity and the influence made by the factories, that is, how to predict the influence on the environment and take the influence as a controlling factor for maintaining the air quality of the environment.

### SUMMARY OF THE DISCLOSURE

The disclosure can be more fully understood by reading the following detailed description of the embodiments, with reference made to the accompanying drawings as described below. It should be noted that the features in the drawings are not necessarily to scale. In fact, the dimensions of the features may be arbitrarily increased or decreased for clarity of discussion.

The present disclosed example is directed to solving the problem of exhausting gas of the factory, and one aspect is to apply the inference method of the causal model, input the sensing data of the gas and refer to the historical sensing data and the weather data of the air-quality monitoring station with applying with the gas dynamics, in order to infer whether the air pollution data sensed by each air-quality monitoring station is influenced by the gas exhausted by the factory and to establish quantitative calibration information of the air-quality monitoring station according to the observation experience or the regression trend.

The present disclosure of an embodiment provides an air pollution forecast management system including an air quality management device and an Internet of Things (IOT) cloud platform. The air quality management device includes a dust particle sensing module, and the dust particle sensing module is disposed on a smoke exhaust flue and configured to sense gas exhausted from the smoke exhaust flue. The IoT cloud platform is communicatively connected with the air quality management device and configured to compute, at a second time after a first time, an exhaust gas set of the gas drifting from the first time to the second time by using currently-observed meteorological data at the second time, receive a plurality of air-pollution sets at a plurality of geographic locations at the second time, compute a plurality of influencing results of the plurality of air-pollution sets respectively associated with the exhaust gas set, and generate a feedback instruction according to at least one of the plurality of influencing results to control gas emission of the smoke exhaust flue.

The present disclosure of an embodiment provides an air pollution forecast management method including controlling gas exhausted from a smoke exhaust flue: computing, at a second time after a first time, an exhaust gas set of the gas drifting from the first time to the second time by using a currently-observed meteorological data: receiving, at the second time, a plurality of air-pollution sets at a plurality of geographic locations: computing a plurality of influencing results of the plurality of air-pollution sets respectively associated with the exhaust gas set: and generating a feedback instruction according to at least one of the plurality of influencing results to control gas emission of the smoke exhaust flue.

The present embodiments of the air quality management device and the air pollution forecast management method provide the gas-exhaustion strategy by using the plant, and the determination is made whether the factory has to reduce the gas exhaustion according to the plurality of influencing results, such that the countermeasures may be introduced before the gas has cumulated in the air, and therefore the trade-off between the economic activity and the environment protection is balanced.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiments, with reference made to the accompanying drawings as described below. It should be noted that the features in the drawings are not necessarily to scale. In fact, the dimensions of the features may be arbitrarily increased or decreased for clarity of discussion.

FIG. 1 is a schematic diagram illustrating the air quality of the monitored environment near the smoke exhaust flue according to one embodiment of the present disclosure.

FIG. 2 is a block diagram illustrating an air pollution forecast management system according to one embodiment of the present disclosure.

FIG. 3 is a schematic diagram illustrating that a smoke exhaust flue of a factory exhausts gas according to one embodiment of the present disclosure.

FIG. 4 is a schematic diagram illustrating a predicted exhaust gas set of the gas exhausted by the smoke exhaust flue of the factory after the gas drifts for a period of time in the future according to one embodiment of the present disclosure.

FIG. 5 is a schematic diagram illustrating a control relation between sensing data and an upper limit of the gas emission according to one embodiment of the present disclosure.

FIG. 6 is a schematic diagram of continuously predicting the gas exhausted by the factory and continuously predicting influencing results of the environment near the factory according to one embodiment of the present disclosure.

FIG. 7 is a schematic diagram illustrating that a predicted gas track of the gas is tagged on a map according to one embodiment of the present disclosure.

FIG. 8 is a schematic diagram illustrating distances, between the micro air-quality monitoring station and the plant, corresponding to sensing data of the micro air-quality monitoring station according to one embodiment of the present disclosure.

FIG. 9 is a schematic diagram illustrating that a grid gas track of the gas is tagged on a map according to one embodiment of the present disclosure.

FIG. 10 is a flow chart illustrating an air pollution forecast management method according to one embodiment of the present disclosure.

FIG. 11 is a flow chart illustrating an air pollution forecast management method according to another embodiment of the present disclosure.

FIG. 12 is a flow chart illustrating an air pollution forecast management method according to another embodiment of the present disclosure.

FIG. 13 is a flow chart illustrating an air pollution forecast management method according to another embodiment of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

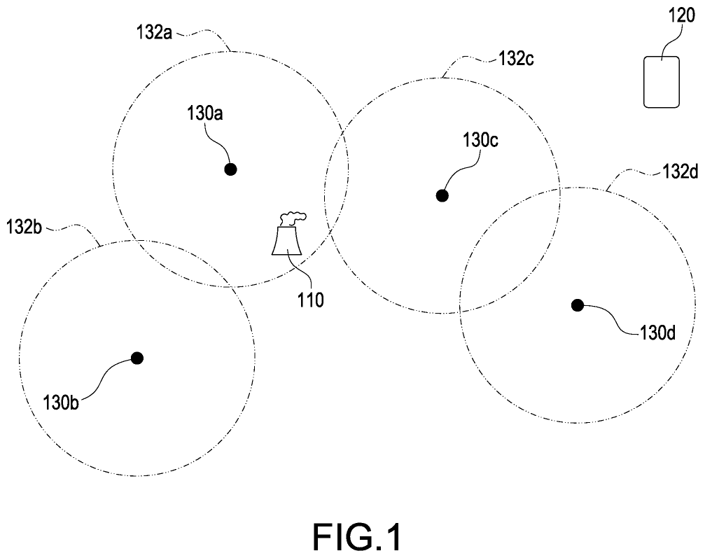

Reference is made to FIG. 1. FIG. 1 is a schematic diagram illustrating the air quality of the monitored environment near the smoke exhaust flue according to one embodiment of the present disclosure. As shown in FIG. 1, a factory 110 is settled in an industrial zone and the smoke exhaust flue of the factory 110 exhausts the gas of different concentrations over time. A local air-quality monitoring station 120 monitors the air quality of a large area (such as 10 kilometers in diameter) and traces the air quality trends in long term. A plurality of micro air-quality monitoring stations 130a to 130d are settled around the factory 110 and monitor the air quality of a small area (such as 300 meters in diameter). In the embodiment of FIG. 1, monitoring areas of the micro air-quality monitoring stations 130a to 130d are sensing areas 132a to 132d respectively. The monitoring area of the local air-quality monitoring station 120 is much larger than the monitoring area of the micro air-quality monitoring stations 130a to 130d. For example, the whole area in FIG. 1 is the sensing area of the local air-quality monitoring station 120, so the sensing area of the local air-quality monitoring station 120 is not drawn especially.

The local air-quality monitoring station 120 and the micro air-quality monitoring stations 130a to 130d are configured to measure the sensing data of particulate matter PM10 or fine particulate matter PM2.5, and each of the air-quality monitoring stations 120, and 130a to 130d are settled at fixed positions (i.e., at a plurality of fixed geographic locations) to periodically monitor the air quality data of the plurality of geographic locations, so the measured air quality data carries coordinates of the plurality of geographic locations and time stamp information correspondingly.

In one embodiment, the amount of data measured by the micro air-quality monitoring stations 130a to 130d per unit time is greater than the amount of data measured by the local air-quality monitoring station 120 per unit time. However, the sensing data of the micro air-quality monitoring stations 130a to 130d may only be used to determine the qualitative trend instead of quantitative trend because they are highly settled among the areas. For this reason, the sensing data of the local air-quality monitoring station 120 is more precise than that of the micro air-quality monitoring stations 130a to 130d, and therefore the present disclosure considers the sensing data of the local air-quality monitoring station 120, the data of the air quality management device 210 of the factory 110, and the sensing data of the micro air-quality monitoring stations 130a to 130d, for analyzing the air quality data, such that overall obtained result is accurate. Furthermore, the local air-quality monitoring station 120 having the calibration ability of quantitative (ISO 17025 standard) and the air quality management device 210 of the factory are used as the basis of quantitive calibration at the local area, and the sensing data of the micro air-quality monitoring stations 130a to 130d is calibrated based on a normal distribution rate to fit the standard of semi-quantitive in accordance with the long term training and result-solving of the air-pollution causal reasoning and atmospheric diffusion model, so as to achieve the purpose of increasing the a period accuracy of the highly settled sensors for qualitative trend by way of the learning pattern. The overall consideration of applying all the data of the air-quality monitoring station is described below.

It should be noted that the local air-quality monitoring station 120 and the micro air-quality monitoring stations 130a to 130d sense the air quality in the sensing area and acquire the sensing data to establish sensing data sets. The sensing data set includes a plurality of sensing data and each of the sensing data includes a coordinate and a timestamp of the present measured time. Because each of the sensing data includes the coordinate and the timestamp, the sensing data sets may provide the time and coordinate information for analyzing the air quality of an indicated time and an indicated area.

On the other hand, the local air-quality monitoring station 120 and the micro air-quality monitoring stations 130a to 130d maintain an air quality classification in advance. The sensing data may be classified based on different intervals into multiple levels, and air-pollution sets are created respectively by the sensing data being classified as same level. Each air-pollution set includes the plurality of sensing data satisfying one interval, and the plurality of sensing data respectively includes the coordinate and the timestamp when the sensing data is measured. Each of the air-quality monitoring stations 120, 130a to 130d continuously monitors the air quality, so the amount of sensing data is large, however, only the sensing data showing the bad air quality is used for analysis. For the sake of brevity, the air-pollution set is used to represent the sensing data that is applied for tracing or warning in the disclosure.

The gas exhausted from the smoke exhaust flue of the factory 110 moves based on the gas dynamics (such as the Brownian's movement). Furthermore, the pollutants become air pollutions (such as sulfur oxides (Sox), carbon monoxide (CO), or nitrogen oxide (NOx)) or particulate pollutions (such as the particulate matter PM10 or fine particulate matter PM2.5) after being released into the air. Air pollution diffuses, settles, and drifts to random areas because of the meteorological factor (such as the wind direction, wind speed, temperature, humidity, and so on) combined with the gas dynamics. In sum, the sensing data of the air-quality monitoring stations 120, and 130a to 130d should be considered to determine the influence caused by the gas exhausted by the factory 110 to the environment.

In the embodiment of FIG. 1, the factory 110 locates within the sensing area of the local air-quality monitoring station 120 and sensing area 132a of the micro air-quality monitoring station 130a, outside the sensing areas 132b, 132c, and 132d of the micro air-quality monitoring stations 130b, 130c, and 130d. It should be noted that the sensing area represents the sensing capability of the air-quality monitoring station, that is, the sensing area represents the largest area where the air-quality monitoring station can monitor the air quality.

Generally, when the air quality sensed by the air-quality monitoring station within the monitoring area is worse, the pollution source may be derived by the gas source most close to the air-quality monitoring station. For example, the micro air-quality monitoring station 130a monitors the bad air quality and the factory 10 is the closest gas source to the micro air-quality monitoring station 130a. In this situation, the factory 110 is regarded as the source that influences the air quality.

In some situations, even if the factory 110 is the gas source closest to the micro air-quality monitoring station 130a, it is not certain that the air-pollution source is the factory 110 because the meteorological factors may affect the air-pollution flow, and other factories (not shown in FIG. 1) far away from the micro air-quality monitoring station 130a may be the air-pollution source instead. The gas exhausted by other factories, having a long distance from the micro air-quality monitoring station 130a, may drift to the micro air-quality monitoring station 130a because the flow of the gas is affected by current wind directions, current wind speeds, current temperatures, and current humidity, or the like, such that the air quality sensed by the micro air-quality monitoring station 130a is not good. The present disclosure may estimate the pollution source that affects the sensing data of the air-quality monitoring station (such as the sensing value becomes greater), by using the gas dynamics and the weather data combining with the Causal Inference Model. The gas exhausted from the factory 110 is the input data of the Causal Inference Model and the influence to the air-quality monitoring station is the output data of the Causal Inference Model. By considering the influence being estimated, the IoT cloud platform 220 may implement that: (a) the factory automatically monitors the exhausting gas and reduces the amount of the exhaustion gas: (b) the deployment of a plurality of air quality management device 210 (that provides a plurality of first sensing data) near the factory 110 and a plurality of local air-quality monitoring stations 120 (that provides a plurality of second sensing data) constructs the mesh nodes forming a three-dimension space (such as X-coordinate, Y-coordinate, and the pollution concentration) within a specific time interval, and a predictor-corrector model is performed to estimate and calibrate a plurality of to-be-calibrated sensing data of the plurality of micro air-quality monitoring stations 130a to 130d nearby to generate the calibrated data of the micro air-quality monitoring station. By calibrating the data of the micro air-quality monitoring stations, the precision of the overall measurement and monitoring ability increases.

In one embodiment, the predictor-corrector model includes and is not limited to a machine learning model, a regression analysis model, an outliner analysis model, a median computation, an average computation, and a normal distribution computation.

The industrial zone is a zone where many factories are settled. For the sake of brevity, one factory (such as the factory 110 in FIG. 1) is taken as an example for illustrating how the factory itself implement monitoring and reducing the gas emissions.

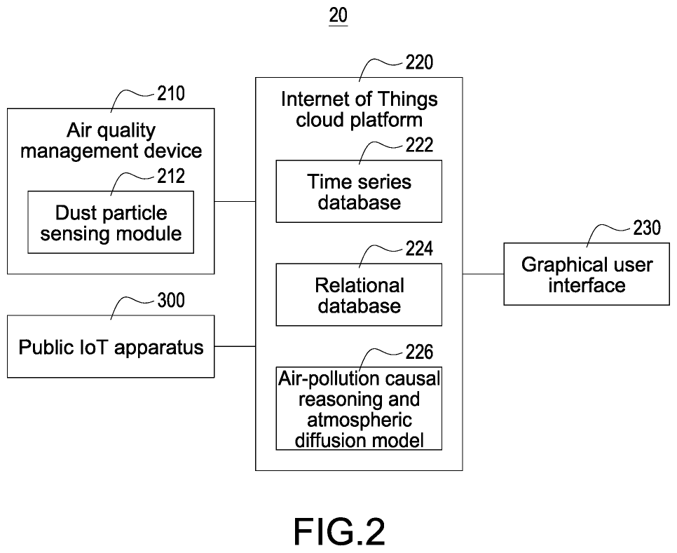

Reference is made to FIG. 2. FIG. 2 is a block diagram illustrating an air pollution forecast management system according to one embodiment of the present disclosure. As shown in FIG. 2, the air pollution forecast management system 20 includes an air quality management device 210, an Internet of Things (IOT) cloud platform 220, a graphical user interface 230, and a public IoT apparatus 300. The IoT cloud platform 220 is connected with the air quality management device 210, the graphical user interface 230, and the public IoT apparatus 300 through a wired manner or a wireless manner.

In one embodiment, the air quality management device 210 includes a dust particle sensing module 212. The dust particle sensing module 212 is disposed on the smoke exhaust flue of the factory 110 and configured to sense the gas exhausted from the smoke exhaust flue. The dust particle sensing module 212 is configured to generate sensing data of the gas, such as total suspended particulate (TSP), PM10, PM2.5, opacity (OP), and the like.

In one embodiment, the IoT cloud platform 220 includes a time series database 222, a relational database 224, and an air-pollution causal reasoning and atmospheric diffusion model 226. The IoT cloud platform 220 receives the sensing data from the air quality management device 210 and the public IoT apparatus 300. However, the time of receiving the sensing data by the air quality management device 210 and by the public IoT apparatus 300 are asynchronous, it is necessary for the IoT cloud platform 220 to sequence the sensing data according to the timestamp of the sensing data and then store the sequenced sensing data in the time series database 222.

In one embodiment, the air-pollution causal reasoning and atmospheric diffusion model 226 of the IoT cloud platform 220 generates a computation result based on the sensing data and the weather data of the last one hour and generates a prediction result for the next 12 hours based on the computation result and real-time sensing data generated at present. The IoT cloud platform 220 stores the computation result and the prediction result in the relational database 224.

In one embodiment, the graphical user interface 230 is configured to display the prediction result, such as the prediction result in the next 12 hours.

In the disclosure, the influence on the environment exerted by the gas is computed in real-time and shows the real-time impact level of the gas on the environment. In one embodiment, the IoT cloud platform 220 uses currently-observed meteorological data at a second time (after a first time) to compute an exhaust gas set of the gas drifting from the first time to the second time, to receive a plurality of air-pollution sets at a plurality of geographic locations at the second time, and to compute a plurality of influencing results of the plurality of air-pollution sets respectively associated with the exhaust gas set. Accordingly, the IoT cloud platform 220 may generate a feedback instruction according to at least one of the pluralities of influencing results to control gas emission of the smoke exhaust flue.

For example, as shown in FIG. 1, the local air-quality monitoring station 120 and the micro air-quality monitoring stations 130a to 130d are respectively disposed at various geographic locations and generate air-pollution sets of their own according to the sensing data received at respective geographic locations. The air-pollution set respectively generated by the local air-quality monitoring station 120 and the micro air-quality monitoring stations 130a to 130d includes the plurality of sensing data satisfying a classification. The plurality of sensing data includes the coordinates and the timestamps, and the coordinates and the timestamps are recorded when the sensing data is generated. It should be noted that the coordinates of the plurality of sensing data is within the geographic locations of the air-quality monitoring station that generates the sensing data, that is, the coordinates reflect the sensing area of the air-quality monitoring station.

In some embodiments, the weather data is received from the weather station and includes the data of wind directions, wind speeds, temperatures, and humidity. In the embodiment, the current time, the current wind direction, the meteorological data of the meteorological, and the historical sensing data are used for predicting the influence range with the weather data and the control equation described as below.

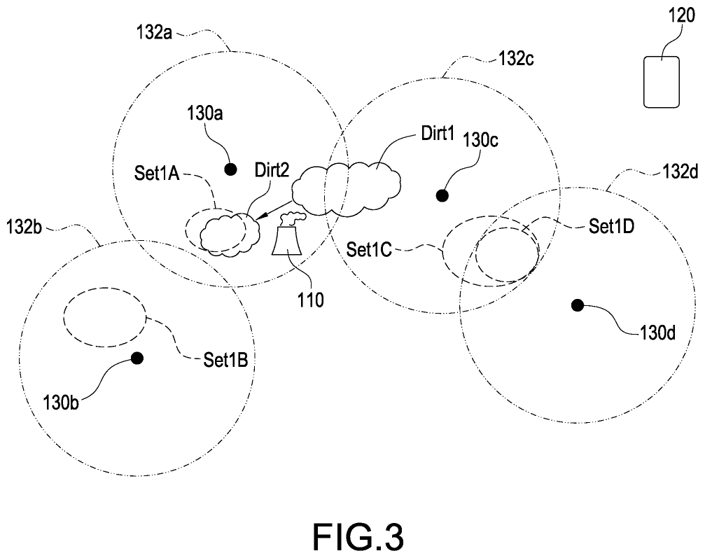

Reference is made to FIG. 3. FIG. 3 is a schematic diagram illustrating that the smoke exhaust flue of the factory exhausts gas according to one embodiment of the present disclosure. As shown in FIG. 3, when the factory 110 exhausts the gas from the smoke exhaust flue, the dust particle sensing module 212 may generate an exhaust gas set Dirt1 at time T1, and the exhaust gas set Dirt1 represents the initial area of the gas exhausted from the smoke exhaust flue to the environment. It should be noted that the dust particle sensing module 212 senses a pollution concentration within a particular area, the exhaust gas set Dirt1 includes one or more sensing data, and the sensing data respectively includes the coordinates and the timestamps when the sensing data is generated. After the gas drifts from time T1 to time T2, the IoT cloud platform 220 computes an exhaust gas set Dirt2 of the gas drifting at time T2 according to the currently observed wind direction at time T2, such as the northeast direction. It should be noted that, because the IoT cloud platform 220 refers to the currently observed weather data, the time interval from time T1 to time T2 is short, such as 10 minutes. The observed weather data received by the IoT cloud platform 220 at time T2 may be the weather data generated at the weather station of the time interval from time T1 to time T2, and time T1 is the past time (e.g., past 10 minutes from now) while time T2 is the current time.

For estimating which surrounding area is influenced by the exhaust gas set Dirt2, the IoT cloud platform 220 receives current air-pollution sets Set1A to Set1D of all the air-quality monitoring stations at time T2 (such as the local air-quality monitoring station 120 and the micro air-quality monitoring stations 130a to 130d in FIG. 3). The IoT cloud platform 220 may compute the influence, made by the exhaust gas set Dirt2, of the sensing data of each air-quality monitoring station by using the sensing data and the coordinates and the timestamps of the current air-pollution sets Set1A to Set1D.

As shown in FIG. 3, at time T2, the current air-pollution set Set1A of the micro air-quality monitoring station 130a is partially overlapped with the exhaust gas set Dirt2, that is, the air-pollution sensed by the micro air-quality monitoring station 130a comes from the gas exhausted by the factory 110, so the plurality of influencing results may be used as the feedback information to notify the factory 110 to reduce the gas emission of the smoke exhaust flue. It should be noted that, in the embodiment, only the sensing data within the sensing area 132a of the micro air-quality monitoring stations 130a is influenced by the exhaust gas set Dirt2. By aerodynamics, in another embodiment, the exhaust gas set Dirt1 may spread into two or more parts at time T2 and become two or more exhaust gas sets that influence two or more respective areas. In this situation, two or more exhaust gas sets induce two or more influencing results, and the computation for two or more exhaust gas sets is similar to that for one exhaust gas set and not repeated herein.

In one embodiment, the IoT cloud platform 220 uses the plume model (such as the Gaussian diffusion model or the control equation) to compute the current pollution concentration/influencing range or the future pollution concentration/influencing range of the gas according to the sensing data continuously sensed from the factory 110, the current weather data, or the future weather data. The description of computing the drifting of the gas according to the weather data to obtain the exhaust gas set is described as below.

In the disclosure, the range of the gas drifting from the factory 110 after a period of time can be predicted to estimate the influence of the surrounding environment in the future, afterwards, the air-pollution source of the surrounding environment may be predicted. In one embodiment, the IoT cloud platform 220 uses, at the second time, the predicted meteorological data of a third time to predict a first predicted exhaust gas set of the exhaust gas set drifting from the second time to the third time, and the third time is a future time while the second time is a current time. The IoT cloud platform 220 uses, at the second time, the predicted meteorological data of the third time to predict a plurality of predicted air-pollution sets of the plurality of air-pollution sets generated by each of the air-quality monitoring stations 120, and 130a to 130d at the third time. The IoT cloud platform 220 computes a plurality of predicted influencing results of the plurality of predicted air-pollution sets respectively associated with the first predicted exhaust gas set to generate a feedback instruction according to at least one of the pluralities of first predicted influencing results.

The predicted meteorological data may be obtained from the weather station, but it is not limited thereto.

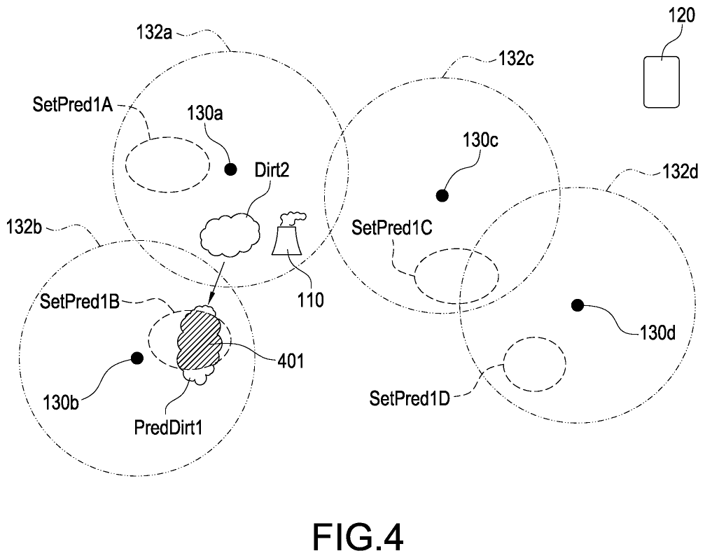

Reference is made to FIG. 4. FIG. 4 is a schematic diagram illustrating a predicted exhaust gas set of the gas exhausted by the smoke exhaust flue of the factory after the gas drifts for a period of time in the future according to one embodiment of the present disclosure. For the sake of understanding, the embodiment of FIG. 4 follows the embodiment of FIG. 3 and illustrates how to compute the predicted exhaust gas sets.

The location and the area of the gas computed by the IoT cloud platform 220 at time T2 is the exhaust gas set Dirt2. In the embodiment, the IoT cloud platform 220 receives the predicted meteorological data of time T3 in advance in order to predict the location and the area of the gas at time T3 that time T3 is after time T2. It should be noted that the time interval between time T2 and time T3 may be 10 minutes, 1 hour, 2 hours, or 12 hours, and it is not limited herein. The predicted meteorological data of time T3 received by the IoT cloud platform 220 at time T2 may be the weather data predicted by the weather station from time T2 to time T3.

If the predicted wind direction is north northeast at time T3, the predicted exhaust gas set PredDirt1 of time T3 may be computed by using the exhaust gas sets, the gas dynamics, and the predicted meteorological data. On the other hand, the IoT cloud platform 220 also predicts the air quality that each air-quality monitoring station (e.g., the micro air-quality monitoring stations 130a to 130d) may sense at time T3. Similarly, the IoT cloud platform 220 predicts, at time T2, by using the air-pollution sets Set1A to Set1D (shown in FIG. 3) of each air-quality monitoring station according to the gas dynamics and the predicted meteorological data to obtain the predicted air-pollution sets SetPred1A to SetPred1D.

As shown in FIG. 4, the IoT cloud platform 220 predicts, at time T2, that the predicted air-pollution set SetPred1B of the micro air-quality monitoring station 130b partially overlaps with the predicted exhaust gas set PredDirt1 at time T3. The IoT cloud platform 220 determines, at time T2, that the air-pollution sensed by the micro air-quality monitoring station 130b at time T3 (in the future) partially comes from the gas drifting from the factory 110, so the predicted influencing result may be referred while the IoT cloud platform 220 generates the feedback information. For example, the factory 110 may be notified by receiving the feedback information for its influence to the surrounding environment at time T3 (the influence in the future) and has to adjust the gas-exhausting strategy.

Similarly, the exhaust gas set Dirt1 may spread into two or more parts in the future and become two or more predicted exhaust gas sets (not shown in FIG. 3), so two or more influencing results may be computed as the similar process described above.

In the disclosure, the gas exhausted from the factory 110 is continuously traced and predicted according to the predicted drifting direction and position, and the influence on the surrounding environment is continuously predicted. In one embodiment, the IoT cloud platform 220 uses, at the second time, the predicted meteorological data of a fourth time to compute a second predicted exhaust gas set of the first predicted exhaust gas set drifting from the third time to the fourth time, computes a plurality of second predicted air-pollution sets of the plurality of first predicted air-pollution set at the fourth time, and computes a plurality of second predicted influencing results of the plurality of second predicted air-pollution sets respectively associated with the second predicted exhaust gas set. In the embodiment, the IoT cloud platform 220 determines whether to generate, at the second time, the feedback instruction according to at least one of the pluralities of second predicted influencing results.

In another embodiment, the IoT cloud platform 220 determines whether to dynamically adjust the gas emission of the factory 110 according to the relation between the sensing data of each of air-quality monitoring stations 120 and 130a to 130d and the upper limit of the gas emission.

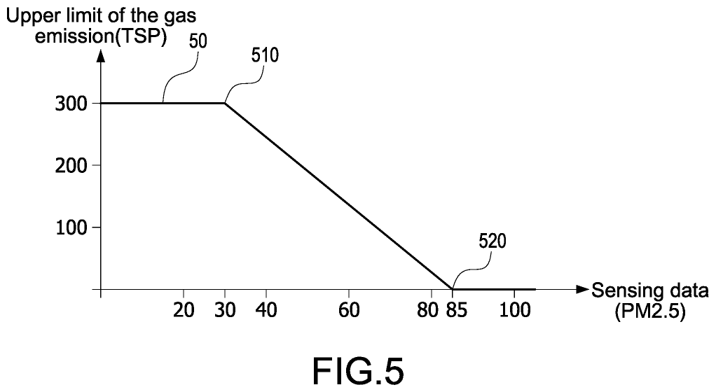

Reference is made to FIG. 5. FIG. 5 is a schematic diagram illustrating a control relation between sensing data and an upper limit of the gas emission according to one embodiment of the present disclosure. The control relation 50 shown in FIG. 5 includes a plurality of control points, and each control point includes the sensing data and the upper limit of the gas emission. The IoT cloud platform 220 determines whether to generate the feedback information according to the control relation 50. The following illustrates the example that the sensing data is PM2.5 (ug/m³) and the upper limit of the gas emission is TSP (ug/m³).

In one example, the PM2.5 value sensed by the micro air-quality monitoring station is 30 ug/m³ and the actual emission from the factory 110 is 150 ug/m³. Because the control point 510 corresponding to the upper limit of TSP is 300 ug/m³ and the actual emission of the factory 110 (e.g., 150 ug/m³) is lower than the upper limit, the IoT cloud platform 220 will not generate the feedback information of requiring the factory 110 to reduce the gas emission. In another example, the PM2.5 value sensed by the micro air-quality monitoring station is 85 ug/m³ and the actual emission from the factory 110 is 100 ug/m³. Because the control point 520 corresponding to the upper limit of TSP is 0 ug/m³, no gas is allowed to be exhausted by the factory 110. In this situation, the IoT cloud platform 220 will generate and transmits the feedback information to the factory 110 to notify the factory 110 that the amount of the gas emission exceeds the upper limit and the gas emission should be stopped.

In other examples, the PM2.5 value sensed by the micro air-quality monitoring station is 30˜85 ug/m³. The acceptable gas emission from the smoke exhaust flue of the factory 110 linearly or gradually reduces from the largest TSP value 300 ug/m³ to 0 ug/m³ along with the increasing of the PM2.5 value. In other words, the larger PM2.5 value the micro air-quality monitoring station senses, the smaller gas amount the factory 110 is allowed to exhaust. When the IoT cloud platform 220 computes and obtains the predicted exhaust gas set and the predicted air-pollution set, the control relation may be applied for notifying the factory 110 earlier, such that the factory 110 may take action on its own in advance.

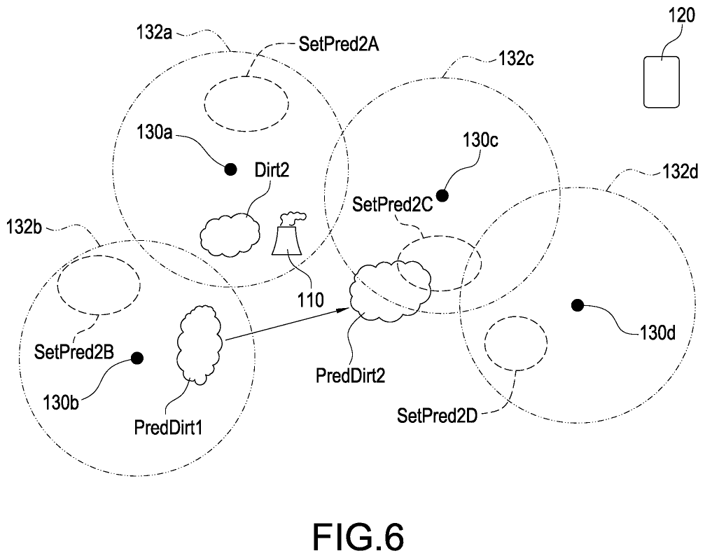

Reference is made to FIG. 6. FIG. 6 is a schematic diagram of continuously predicting the gas exhausted by the factory and continuously predicting influencing results of the environment near the factory according to one embodiment of the present disclosure. For the sake of understanding, the embodiment in FIG. 6 follows the embodiments in FIG. 3 and FIG. 4 and is illustrated below.

In the embodiment, the IoT cloud platform 220 predicts, at time T2, the location and the area of the gas of time T3, and the predicted location and the area of the gas of time T3 is the predicted exhaust gas set PredDirt1. Similarly, the IoT cloud platform 220 receives the predicted meteorological data of time T4 in advance in order to predict the location and the area of the gas of time T4 that time T4 is after time T3. It should be noted that the time interval between time T3 and time T4 may be 10 minutes, 1 hour, 2 hours, or 12 hours, and it is not limited herein. The predicted meteorological data of time T4 received by the IoT cloud platform 220 at time T2 may be the weather data predicted by the weather station from time T3 to time T4 or the time before time T4.

If the predicted wind direction is west southwest at time T4, the predicted exhaust gas set PredDirt2 of time T4 may be computed by using the predicted exhaust gas set PredDirt1, the gas dynamics, and the predicted meteorological data. Similarly, the IoT cloud platform 220 predicts, at time T3, by using the predicted air-pollution sets PredSet1A to PredSet1D of each air-quality monitoring station according to the gas dynamics and the predicted meteorological data to obtain the predicted air-pollution sets SetPred2A to SetPred2D of each air-quality monitoring station at time T4.

As shown in FIG. 6, the IoT cloud platform 220 predicts, at time T2, that the predicted air-pollution set SetPred2C of the micro air-quality monitoring station 130c partially overlaps with the predicted exhaust gas set PredDirt2 at time T4. That is, the IoT cloud platform 220 determines, at time T2, that the air-pollution sensed by the micro air-quality monitoring station 130c at time T4 (in the future) partially comes from the gas drifting from the factory 110, so the predicted influencing result may be referred while the IoT cloud platform 20 generates the feedback information. For example, the factory 110 may be notified by receiving the feedback information for the air-pollution influence of time T4 (the influence in the future) on the surrounding environment and has to adjust the gas-exhausting strategy immediately. The IoT cloud platform 220 may also use the control relation 50 in FIG. 5 to generate the feedback instruction and the illustration is not repeated.

It should be noted that time T1, T2, T3, and T4 may be the time series with same or different intervals. The terms “time T1”, “time T2”, “time T3”, and “time T4” are exchangeably used with “time point T1”, “time point T2”, “time point T3”, and “time point T4”.

The embodiment of FIG. 3 illustrates how to compute, at time T2, the drifting area of the gas by using the known exhaust gas set and the currently observed weather data. The embodiment of FIG. 4 illustrates how to predict, at time T2, the exhaust gas set of time T3 by using the known exhaust gas set and the predicted meteorological data. The embodiment of FIG. 6 illustrates how to predict, at time T2, the future drifting area of the gas by using the predicted exhaust gas set according to the predicted meteorological data. In the embodiment, the disclosure provides the technical feature of predicting the future drifting area of the gas based on the recurrence, such that the total predicted time length of the future drifting area may be increased.

In another embodiment, the IoT cloud platform 220 may receive, at time T3, the actual weather data sensed by the weather station and determine whether to calibrate the predicted air-pollution sets PredSet1A to PredSet1D, that have been predicted at time T2, with respect to the air-pollution sets Set1A to Set1D of the micro air-quality monitoring stations 130a to 130d, so as to generate the calibrated gas area and the calibrated air-pollution set. The calibration is similar to the illustration of FIG. 3, that is, the drafting area is computed by the exhaust gas set and the currently-observed meteorological data, and the description is not repeated. In one embodiment, the IoT cloud platform 220 may compute, at time T3, the plurality of influencing results again according to the calibrated gas area and the calibrated air-pollution set, and generate a calibration instruction. Therefore, the accuracy of the information provided to the factory 110 is increased.

In the disclosure, the gas exhausted from the factory 110 is continuously traced and predicted according to the predicted drifting direction and position, so the gas track is obtained. In one embodiment, the IoT cloud platform 220 obtains a predicted gas track of the gas drifting from the second time to the fourth time according to the exhaust gas set of the second time, the first predicted exhaust gas set of the third time, and the second predicted exhaust gas set of the fourth time, and tags the predicted gas track on the map.

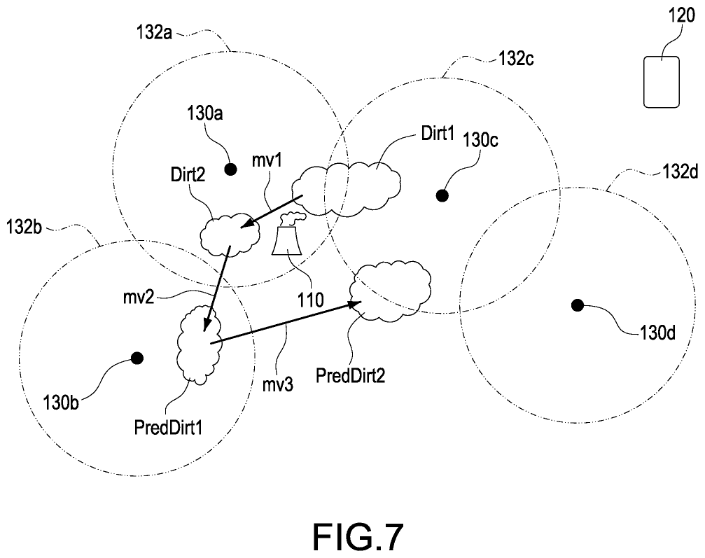

Reference is made to FIG. 7. FIG. 7 is a schematic diagram illustrating that a predicted gas track of the gas is tagged on a map according to one embodiment of the present disclosure. As illustrated in FIG. 7, the areas of the gas drifting at time T1 and time T2 by the factory 110 are the exhaust gas set Dirt1 and the exhaust gas set Dirt2 respectively, and the IoT cloud platform 220 obtains a moving path mv1 according to the exhaust gas set Dirt1 and the exhaust gas set Dirt2. The area of the gas drifting at time T2 is the exhaust gas set Dirt2 and the predicted area of the gas at time T3 is a predicted exhaust gas set PredDirt1, so the IoT cloud platform 220 obtains a moving path mv2 according to the exhaust gas set Dirt2 and the predicted exhaust gas set PredDirt1. The predicted areas of the gas drifting at time T3 and time T4 are the predicted exhaust gas set PredDirt1 and the predicted exhaust gas set PredDirt2 respectively, so the IoT cloud platform 220 obtains a moving path mv3 according to the predicted exhaust gas set PredDirt1 and the predicted exhaust gas set PredDirt2. Accordingly, the IoT cloud platform 220 may obtain the predicted gas track of the gas, and the predicted gas track of the gas includes the moving path mv1, the moving path mv2, and the moving path mv3.

The predicted gas track of the gas described above is tagged on the map by the IoT cloud platform 220 (such as displayed on the monitor of the supervisory system or the mobile device of the administrator of the factory 110), such that the administrator of the factory 110 may refer the information to determine whether to adjust the gas emission of the factory 110. In another embodiment, the IoT cloud platform 220 executes the iterated computation to predict a future long-term trend of the gas (such as over 12 hours). For the sake of brevity of the figure, the IoT cloud platform 220 only tags the predicted gas track having 10 kilometers (for a cumulative duration) in the scale of length on the map.

As described above, by computing the area of the gas drifting based on the plume model and computing the predicted air-pollution set of each air-quality monitoring station in advance, the gas-influenced level of the predicted air-pollution set in each air-quality monitoring station is computed accordingly.

In one embodiment, the IoT cloud platform 220 computes a plurality of coverage distribution proportions that the first predicted exhaust gas set respectively covers the plurality of predicted air-pollution sets, and the plurality of coverage distribution proportions are used as the plurality of first predicted influencing results. When at least one of the pluralities of coverage distribution proportions is greater than a distribution threshold, the IoT cloud platform 220 generates the feedback instruction, and the feedback instruction is used to control the smoke exhaust flue of the factory 110 to gradually reduce the gas emission.

Because each sensing data carries the location information (such as the coordinate) of its own, the IoT cloud platform 220 may compare the location information of the predicted exhaust gas set with the location information of the predicted air-pollution set of each micro air-quality monitoring station to calculate the overlapped area, and the overlapped area is used as the gas coverage area of predicted exhaust gas set of each micro air-quality monitoring station. The IoT cloud platform 220 computes the gas coverage area corresponding to the area of the predicted air-pollution set of each micro air-quality monitoring station to obtain the coverage distribution proportion.

For example, as shown in FIG. 4, the IoT cloud platform 220 compares the location information of the predicted exhaust gas set PredDirt1 with the location information of the predicted air-pollution sets PredSet1A to PredSet1D respectively to obtain an overlapped area 401. The IoT cloud platform 220 computes the overlapped area 401 corresponding to the area of the predicted air-pollution set PredSet1B to obtain a coverage distribution proportion, e.g., 50%, of the predicted exhaust gas set PredDirt1 covering the predicted air-pollution set PredSet1B. In the embodiment, the predicted exhaust gas set PredDirt1 does not overlap with the predicted air-pollution sets PredSet1A, PredSet1C, and PredSet1D, so the coverage distribution proportions of the predicted air-pollution sets PredSetA, PredSet1C, and PredSet1D are 0%.

In another embodiment, when multiple factories are settled near same micro air-quality monitoring station (such as within an area of a circle with a diameter of 10 kilometers), the IoT cloud platform 220 computes respectively the micro air-quality monitoring station influenced by the multiple factories to obtain multiple influenced ratios. The target factory is determined by the largest influenced ratio, and the feedback instruction is transmitted to the target factory. Taking three factories settled within the area near same micro air-quality monitoring station as an example, the IoT cloud platform 220 computes respectively the ratio of the micro air-quality monitoring station influenced by a first factory, a second factory, and a third factory, and obtains the ratio 20%, 15%, and 10% respectively. The IoT cloud platform 220 determines that the factory having the largest ratio, i.e., 20%, is the factory that influences the micro air-quality monitoring station the most, i.e., the first factory. In one embodiment, the feedback instruction generated by the IoT cloud platform 220 is transmitted to the first factory.

In another embodiment, the IoT cloud platform 220 computes at least one coverage distribution of the first predicted exhaust gas set with respect to the plurality of predicted air-pollution sets, and determines whether a pollution concentration among the at least one coverage distribution is greater than a concentration threshold. When the IoT cloud platform 220 determines that the pollution concentration is greater than the concentration threshold, the feedback instruction is generated and used to control the smoke exhaust flue to decrease the gas emission gradually.

For example, as shown in FIG. 4, the IoT cloud platform 220 computes the coverage that the exhaust gas set PredDirt1 corresponds to the micro air-quality monitoring station 130b (such as the overlapped area) according to the coordinates of the sensing data of the predicted exhaust gas set PredDirt1. If the IoT cloud platform 220 determines that the pollution concentration of the coverage of the micro air-quality monitoring station 130b (such as the average of the pollution concentration, the largest value of the pollution concentration, the median number of the pollution concentration, and so on) is greater than the threshold, the IoT cloud platform 220 generates and transmits the feedback instruction to the factory 110 for reducing the gas emission.

It should be noted that the IoT cloud platform 220 may compute the plurality of coverage distribution proportions that a second predicted exhaust gas set (such as the predicted exhaust gas setPredDirt2 in FIG. 6) respectively covers the plurality of predicted air-pollution sets to determine whether to generate the feedback instruction. The IoT cloud platform 220 also computes at least one coverage that the second predicted exhaust gas set (such as the predicted exhaust gas setPredDirt2 in FIG. 6) respectively covers the plurality of second predicted air-pollution sets. Therefore, the IoT cloud platform 220 generates the feedback instruction when determining that the pollution concentration of the at least one coverage is greater than the concentration threshold. The detailed is similar to the above description and not repeated.

Generally, the amount of the micro air-quality monitoring stations 130a to 130d is relatively more and the amount of the local air-quality monitoring station 120 is relatively less; however, the data precision of the micro air-quality monitoring stations 130a to 130d is lower than that of the local air-quality monitoring station 120. Furthermore, data error or data bias exits among the micro air-quality monitoring stations 130a to 130d due to their hardware differentiation. If the sensing data sets (including all the past sensing data) of the micro air-quality monitoring stations 130a to 130d are directly inputted into the computation process, an incorrect computation result may be generated due to the data error or data bias mentioned above. In one embodiment, before performing computations and prediction processes, calibration for the sensing data sets of the micro air-quality monitoring station 130a to 130d is required.

In one embodiment, before the IoT cloud platform 220 performs the computations and prediction processes, the outlier computation is performed to remove non-correlated data from the plurality of sensing data of the air-pollution set of the micro air-quality monitoring stations 130a to 130d to obtain a plurality of historical sensing data calibration set. The IoT cloud platform 220 uses the predicted meteorological data and data of the plurality of historical sensing data calibration sets to perform the computations and prediction processes to obtain the plurality of predicted air-pollution sets.

Because dust or particulates diffuse and settle in the air, their movements in the air differ from their particle size (e.g., the time length the particles stay or the distance the particles travel). A distance exists between the factory 110 (which is regarded as the starting point) and the micro air-quality monitoring station and the sensing data of the micro air-quality monitoring station differs along with the distance. For example, different micro air-quality monitoring stations having same or similar distance with the factory 110 may have the sensing data of same or similar feature value with each other. In one embodiment, the sensing data that is too far away from the feature value group is filtered so that the sensing data sets of each micro air-quality monitoring station are calibrated.

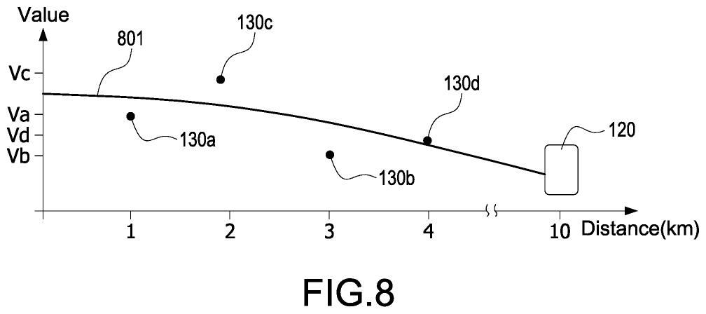

Reference is made to FIG. 8. FIG. 8 is a schematic diagram illustrating distances, between the micro air-quality monitoring station and the factory, corresponding to sensing data of the micro air-quality monitoring station according to one embodiment of the present disclosure. As shown in FIG. 8, the distance between the micro air-quality monitoring stations 130a and the factory 110 is 1 kilometer, and the sensing data of the micro air-quality monitoring station 130a is represented by the value Va. Similarly, the distances between the factory 110 and the micro air-quality monitoring stations 130b, 130c, and 130d are 3 kilometers, 2 kilometers, and 4 kilometers respectively, and the sensing data is represented by the value Vb, Vc, and Vd respectively. The distance is the straight-line distance but is not limited herein.

The sensing data of each of the micro air-quality monitoring stations 130a to 130d, the local air-quality monitoring station 120, and the factory 110 is stored as the historical sensing data sets, and the IoT cloud platform 220 calibrates the historical sensing data sets of each micro air-quality monitoring station by using the data feature of the sensing data of the historical sensing data sets. For example, the IoT cloud platform 220 normalizes and denormalizes the historical sensing data sets of the micro air-quality monitoring stations 130a to 130d, the local air-quality monitoring station 120, and the factory 110 according to the distance between the factory 110 and each of the local air-quality monitoring station 120 and the micro air-quality monitoring stations 130a to 130d to obtain the linear relation or non-linear relation between the distance and the value of the sensing data, shown as curve 801 in FIG. 8.

The IoT cloud platform 220 determines whether the value of the sensing data of each of the micro air-quality monitoring stations 130a to 130d deviates from the curve 801 too much or computes the value of the sensing data by the machine learning algorithm to determine the deviation. If the IoT cloud platform 220 determines that the value of any sensing data deviates from the curve 801, the sensing data is excluded and the remaining sensing data is used as the data of the historical sensing data calibration set. After the sensing data sets of each of the micro air-quality monitoring stations are calibrated by the IoT cloud platform 220, the historical sensing data calibration set of each of the micro air-quality monitoring stations is obtained. The IoT cloud platform 220 uses the predicted meteorological data and the historical sensing data calibration set to compute the predicted air-pollution set that is described above and will be omitted here. It should be noted that no sensing data is deleted from the air-quality monitoring station. For generating the historical sensing data calibration set, the sensing data with the data feature satisfying the requirement is collected to be the historical sensing data calibration set for the following computation.

In the disclosure, the unreasonable outlier is excluded from the historical sensing data to collect the remaining sensing data to be the data of the historical sensing data calibration set, such that the nature of the sensing data transforms from qualitative into semi-quantitative, so that all the data of the historical sensing data calibration set becomes valuable.

In another embodiment, the IoT cloud platform 220 determines whether the values sensed by the air-quality monitoring station have a relation with the gas exhausted by the factory 110 by using the Sensitivity analysis. For example, when the gas emission of the factory 110 increases on a vast scale compared to the normal condition and all the values of the sensing data of the air-quality monitoring station near the factory 110 increase as well, it represents that all the sensing data has a positive correlation with the gas exhausted by the factory 110 even if the data of the micro air-quality monitoring station is less precise than the data of the local air-quality monitoring station, they still have same variation trend. At this scenario, the IoT cloud platform 220 uses all the sensing data of each air-quality monitoring station to perform the analysis.

In one embodiment, each weather data carries the timestamp and the geographic location, and the geographic location is the geographic location of the weather station. The gas drifts in the air following the weather condition, however, the number of the weather stations is limited, and it is impossible to predict the drifting direction of the gas at the locations without settling the weather station. In the disclosure, the grid computation is applied to the current weather data (including the currently-observed meteorological data and the predicted meteorological data) to extend the fixed number of the plurality of geographic locations (such as the number of existing weather stations) to other locations without the weather stations. The existing weather data is applied to generate estimated weather data for the locations without settling weather stations, so that the plurality of geographic locations without the weather stations may provide weather data correspondingly.

In one embodiment, both the currently-observed meteorological data and the predicted meteorological data have a first quantity of the geographic location coordinates. The IoT cloud platform 220 performs a grid computation on the currently-observed meteorological data and the predicted meteorological data to extend the currently-observed meteorological data and the predicted meteorological data from the first quantity to a second quantity to obtain the currently-observed meteorological grid data and the predicted meteorological grid data. Therefore, both the currently-observed meteorological grid data and the predicted meteorological grid data have the second quantity of the geographic location coordinates and the predicted meteorological grid data.

For example, four weather stations are respectively settled at 4 different geographic locations, and the four weather stations generate the plurality of weather data and the coordinates of the weather data belong to any one of the 4 geographic locations. By performing the grid computation, the IoT cloud platform 220 may generate 16 virtual weather data among the 4 geographic locations and extend the weather data of 4 geographic locations to the meteorological grid data of 20 geographic locations. The IoT cloud platform 220 performs the grid computation on the currently-observed meteorological data to generate the currently-observed meteorological grid data having the plurality of coordinates, and also performs the grid computation on the predicted meteorological data to generate the predicted meteorological grid data having the plurality of coordinates.

In one embodiment, the IoT cloud platform 220 uses, at the first time, the currently-observed meteorological grid data to compute a first grid exhaust gas set of the gas drifting from the first time to the second time, uses the predicted meteorological grid data to compute a second grid exhaust gas set of the gas drifting from the second time to the third time, obtains a grid gas track of the gas drifting from the first time to the third time according to the exhaust gas set, the first grid exhaust gas set, and the second grid exhaust gas set, and tags the grid gas track on the map, and a resolution of the grid gas track is greater than the resolution of the predicted gas track on the map.

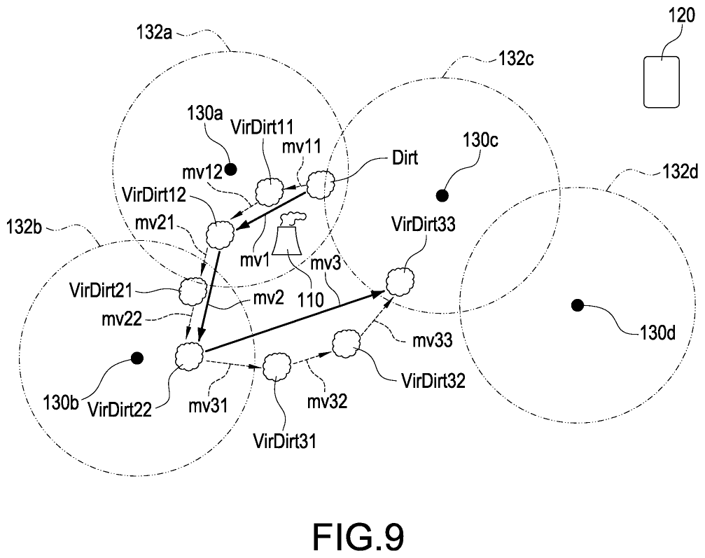

Reference is made to FIG. 9. FIG. 9 is a schematic diagram illustrating that the grid gas track of the gas is tagged on a map according to one embodiment of the present disclosure. As shown in FIG. 9, the IoT cloud platform 220 uses, at time T2, the currently-observed meteorological grid data of time T1 to compute the grid exhaust gas sets VirDirt11 and VirDirt12 of the gas drifting from time T1 to time T2, uses the predicted meteorological grid data of time T3 to compute the grid exhaust gas sets VirDirt21 and VirDirt22 of the grid exhaust gas set VirDirt12 drifting from time T2 to time T3, and uses the predicted meteorological grid data of time T4 to compute the grid exhaust gas sets VirDirt31, VirDirt32, and VirDirt33 of the grid exhaust gas set VirDirt22 drifting from time T3 to time T4. In the embodiment, the IoT cloud platform 220 obtains the grid gas track of the gas, and the grid gas track includes grid moving paths mv11, mv12, mv21, mv22, mv31, mv32, and mv33.

In the embodiment of FIG. 9, because real weather data exist near the micro air-quality monitoring stations 130a and 130b, the grid moving paths mv11 and mv12 do not depart from the moving path mv1 (or only departs from the moving path mv1 within a tolerance) and the grid moving paths mv21 and mv22 do not depart from the moving path mv2 (or only departs from the moving path mv2 within a tolerance).

On the other hand, because an area without real weather data (i.e., has no physical weather stations) exists between the micro air-quality monitoring stations 130b and 130c, the moving path mv3 of the predicted gas track is long and the precision is slightly low.

After executing the interpolation computation (e.g., the grid computation) to obtain the meteorological grid data within the area(s) lacking of real weather data, the IoT cloud platform 220 obtains the grid exhaust gas sets VirDirt31, VirDirt32, and VirDirt33 based on the meteorological grid data, and the grid gas track between the micro air-quality monitoring stations 130b and 130c is obtained, and the grid gas track includes the grid moving paths mv31, mv32, and mv33. Because the number of the grid moving paths forming the grid gas track is greater than that forming the predicted gas track, the grid gas track having a finer resolution is obtained, such that the drifting track of the gas is more precise.

After the IoT cloud platform 220 performs the grid computation, the weather data of the geographic location coordinates fills the blank, such that the drifting direction of the gas tracked by the IoT cloud platform 220 is consecutive without discontinuity due to the lack of the weather data of some geographic locations. In the embodiment, the integrity of the predicted gas track is preserved.

The formats or types of the sensing data differ from a hardware or system environment of the air-quality monitoring stations, so the formats or the types of the sensing data of each air-quality monitoring station may be different. The normalization is applied to the sensing data of different air-quality monitoring stations, so the formats or the types of all the sensing data are consistent with each other to avoid the problem occurring from the different formats or types.

In one embodiment, when determining that the data type of the sensing data of the exhaust gas set is different from that of the air-pollution set, the IoT cloud platform 220 transforms the type of the sensing data of the exhaust gas set into the type of the sensing data of the air-pollution set. In another embodiment, the IoT cloud platform 220 transforms the type of the sensing data of the air-pollution set into the type of the sensing data of the exhaust gas set, and it is not limited herein.

For example, please refer back to FIGS. 2-3, the type of the sensing data sensed by the dust particle sensing module 212 is TSP (total suspended particulate) and the type of the sensing data sensed by the local air-quality monitoring station 120 and the micro air-quality monitoring stations 130a to 130d is PM2.5. The IoT cloud platform 220 computes a converting value for transforming the TSP value to the PM2.5 value according to the geographic location of the historical data, the season of the historical data, or the industry category of the historical data (for example, TSP*0.176=PM2.5, and the converting value is 0.176.) In one embodiment, the type of the sensing data is unified by applying the normalization computation and the sensing data is processed based on same data type, such that the accuracy and precision of computations are increased because the calculation error is prevented from using different types of the sensing data.

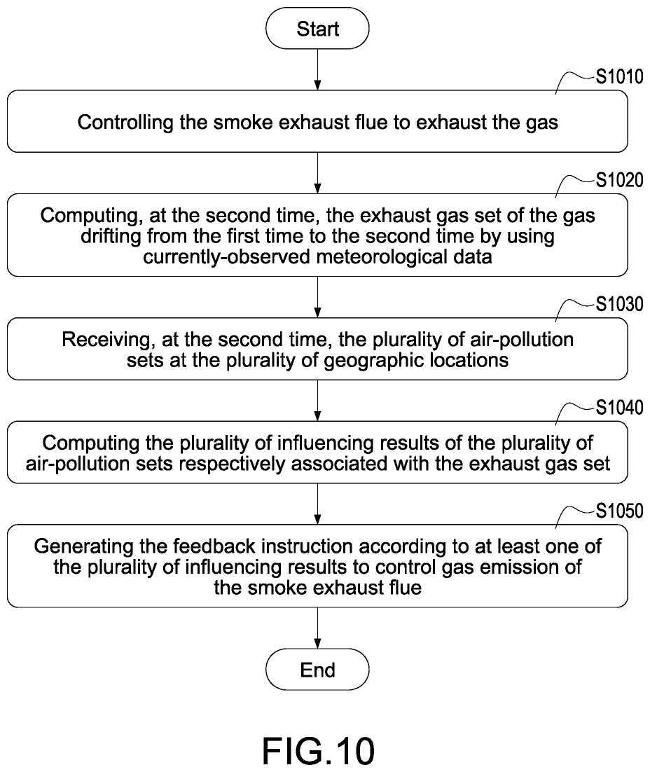

Reference is made to FIG. 10. FIG. 10 is a flow chart illustrating an air pollution forecast management method according to one embodiment of the present disclosure. The air pollution forecast management method is performed by the IoT cloud platform 220 in FIG. 2. Specifically, each step of the air pollution forecast management method is recorded as a program including instructions, and the instructions are stored in the non-transitory computer-readable storage medium. The IoT cloud platform 220 performs the instructions stored in the non-transitory computer-readable storage medium to cause the air pollution forecast management method to be executed.

In step S1010, controlling the smoke exhaust flue to exhaust the gas by the IoT cloud platform 220 is performed.

In step S1020, computing, by the IoT cloud platform 220 at the second time, the exhaust gas set of the gas drifting from the first time to the second time by using currently-observed meteorological data is performed. The first time is before the second time.

In step S1030, receiving, by the IoT cloud platform 220 at the second time, the plurality of air-pollution sets at the plurality of geographic locations is performed.

In step S1040, computing, by the IoT cloud platform 220, the plurality of influencing results of the plurality of air-pollution sets respectively associated with the exhaust gas set is performed.

In step S1050, generating, by the IoT cloud platform 220, the feedback instruction according to at least one of the pluralities of influencing results to control gas emission of the smoke exhaust flue is performed.

Accordingly, the drifting direction of the gas is tracked in real-time, and the gas exhausted by the factory 110 is monitored at the beginning, such that the accuracy of monitoring the drifting direction of the gas is increased.

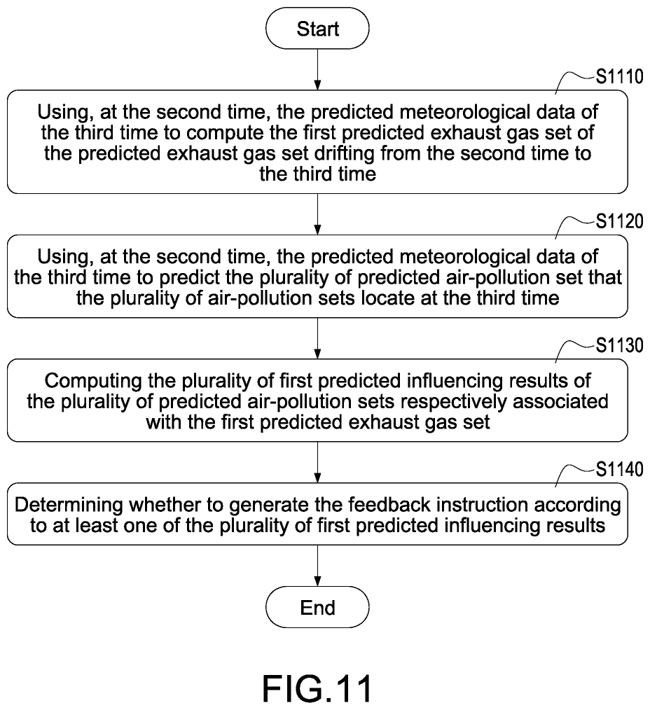

Reference is made to FIG. 11. FIG. 11 is a flow chart illustrating an air pollution forecast management method according to another embodiment of the present disclosure. In one embodiment, the air pollution forecast management method is performed by the IoT cloud platform 220 in FIG. 2.

In step S1110, using, by the IoT cloud platform 220 at the second time, the predicted meteorological data of the third time to compute the first predicted exhaust gas set of the predicted exhaust gas set drifting from the second time to the third time is performed.

In step S1120, using, by the IoT cloud platform 220 at the second time, the predicted meteorological data of the third time to predict a plurality of first predicted air-pollution sets that the plurality of air-pollution sets locate at the third time is performed.

In step S1130, computing, by the IoT cloud platform 220, the plurality of first predicted influencing results of the plurality of first predicted air-pollution sets respectively associated with the first predicted exhaust gas set is performed.

In step S1140, determining, by the IoT cloud platform 220, whether to generate the feedback instruction according to at least one of the pluralities of first predicted influencing results is performed.

In one embodiment, the step of determining whether to generate the feedback instruction of the air pollution forecast management method includes computing the plurality of coverage distribution proportions of the first predicted exhaust gas set respectively covered on the plurality of first predicted air-pollution sets to be the plurality of first predicted influencing results, the plurality of coverage distribution proportions are used as the plurality of first predicted influencing results. When at least one of the pluralities of coverage distribution proportions is greater than the distribution threshold, the feedback instruction is generated.

In another embodiment, the step of determining whether to generate the feedback instruction according to at least one of the plurality of first predicted influencing results of the air pollution forecast management method includes computing at least one coverage distribution respectively between the first predicted exhaust gas set and each of the plurality of first predicted air-pollution sets, determining whether the pollution concentration among the at least one coverage distribution is greater than a concentration threshold, and generating the feedback instruction when the pollution concentration is greater than the concentration threshold.

In one embodiment, the feedback instruction is used to control the smoke exhaust flue to decrease the gas emission gradually from the second time.

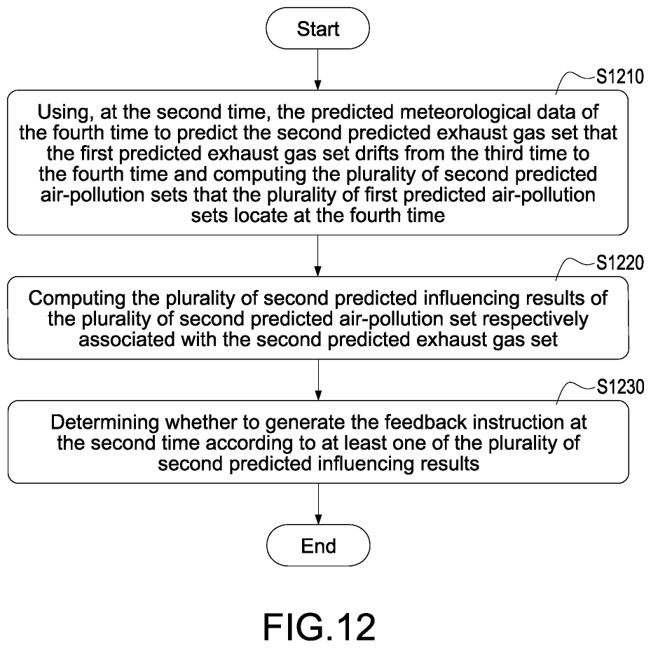

Reference is made to FIG. 12. FIG. 12 is a flow chart illustrating an air pollution forecast management method according to another embodiment of the present disclosure. The air pollution forecast management method is performed by the IoT cloud platform 220 of FIG. 2.

In step S1210, using, by the IoT cloud platform 220 at the second time, the predicted meteorological data of the fourth time to predict the second predicted exhaust gas set that the first predicted exhaust gas set drifts from the third time to the fourth time and computing the plurality of second predicted air-pollution sets that the plurality of first predicted air-pollution sets locate at the fourth time is performed.

In step S1220, computing, by the IoT cloud platform 220, the plurality of second predicted influencing results of the plurality of second predicted air-pollution set respectively associated with the second predicted exhaust gas set is performed.

In step S1230, determining, by the IoT cloud platform 220, whether to generate the feedback instruction at the second time according to at least one of the pluralities of second predicted influencing results is performed.

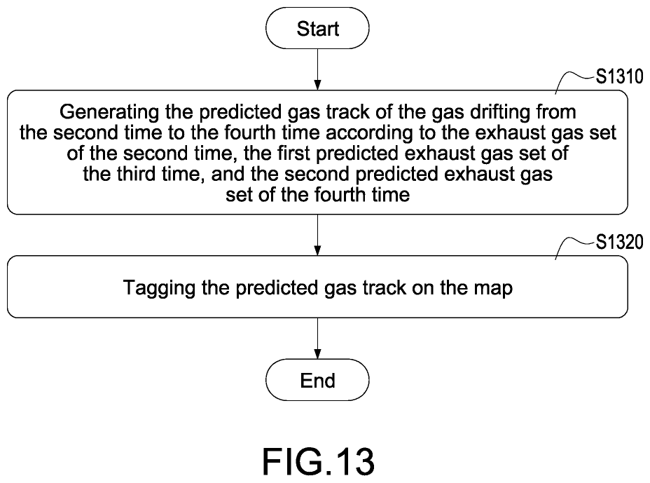

Reference is made to FIG. 13. FIG. 13 is a flow chart illustrating an air pollution forecast management method according to another embodiment of the present disclosure. The air pollution forecast management method is performed by the IoT cloud platform 220 of FIG. 2.

In step S1310, generating, by the IoT cloud platform 220, the predicted gas track of the gas drifting from the second time to the fourth time according to the exhaust gas set of the second time, the first predicted exhaust gas set of the third time, and the second predicted exhaust gas set of the fourth time is performed.

In step S1320, tagging, by the IoT cloud platform 220, the predicted gas track on the map is performed.

In some embodiments, the air pollution forecast management method may apply the gas exhausted by more than one factory to determine the air quality of the environment or the influence of the surroundings.

In one embodiment, the IoT cloud platform 220 uses the currently-observed meteorological data to compute the exhaust gas set of the gas exhausted by a first gas-exhausting source from the first time to the second time and compute the second exhaust gas set of the gas exhausted by a second gas-exhausting source from the first time to the second time, receives an air-pollution set at the plurality of geographic locations at the second time, and respectively computes the influence area or coverage ratio of the first exhaust gas set associated with the air-pollution set and the influence area or coverage ratio of the second exhaust gas set associated with the air-pollution set. The IoT cloud platform 220 generates the feedback instruction to control the gas emission of the gas-exhausting source having the largest influence area or the coverage ratio.

Accordingly, the air quality management device and the air pollution forecast management method in the specification may predict the influence level and the effects on the environment that the sensing data of the micro air-quality monitoring station is affected by the factory in the future, such that the factory may realize, by reference to the predicted influence level, the bad influence on the environment if the factory keeps exhausting the gas, and the factory may make the countermeasure to follow the environmental, social, and governance criteria (ESG).

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An air pollution forecast management system, comprising:
an air quality management device, comprising a dust particle sensing module being disposed on a smoke exhaust flue and configured to sense gas exhausted from the smoke exhaust flue; and
an Internet of Things (IOT) cloud platform, communicatively connected with the air quality management device and configured to compute, at a second time after a first time, an exhaust gas set of the gas drifting from the first time to the second time by using currently-observed meteorological data at the second time, receive a plurality of air-pollution sets at a plurality of geographic locations at the second time, compute a plurality of influencing results of the plurality of air-pollution sets respectively associated with the exhaust gas set, and generate a feedback instruction according to at least one of the plurality of influencing results to control gas emission of the smoke exhaust flue.

2. The air pollution forecast management system of claim 1, wherein, before computing the plurality of influencing results of the plurality of air-pollution sets associated with the exhaust gas set, the IoT cloud platform is configured to execute an outlier computation to remove non-correlated data from a plurality of sensing data to obtain a plurality of historical sensing data calibration sets, and the IoT cloud platform is configured to obtain a plurality of predicted air-pollution sets by using predicted meteorological data and data of the plurality of historical sensing data calibration sets.

3. The air pollution forecast management system of claim 1, wherein the IoT cloud platform is configured to use, at the second time, predicted meteorological data of a third time to compute a first predicted exhaust gas set of the exhaust gas set drifting from the second time to the third time, compute a plurality of first predicted air-pollution set that the plurality of air-pollution sets locate at the third time, and compute a plurality of first predicted influencing results of the plurality of first predicted air-pollution sets respectively associated with the first predicted exhaust gas set to generate the feedback instruction according to at least one of the plurality of first predicted influencing results.

4. The air pollution forecast management system of claim 3, wherein the IoT cloud platform is configured to use, at the second time, the predicted meteorological data of a fourth time to compute a second predicted exhaust gas set that the first predicted exhaust gas set drift from the third time to the fourth time, compute a plurality of second predicted air-pollution sets that the plurality of first predicted air-pollution sets locate at the fourth time, and compute a plurality of second predicted influencing results of the plurality of second predicted air-pollution sets respectively associated with the second predicted exhaust gas set to determine whether to generate the feedback instruction at the second time according to at least one of the plurality of second predicted influencing results.

5. The air pollution forecast management system of claim 4, wherein the IoT cloud platform is configured to obtain a predicted gas track of the gas drifting from the second time to the fourth time according to the exhaust gas set, the first predicted exhaust gas set, and the second predicted exhaust gas set, and the IoT cloud platform is configured to tag the predicted gas track on a map.

6. The air pollution forecast management system of claim 3, wherein the IoT cloud platform is configured to compute a plurality of coverage distribution proportions of the first predicted exhaust gas set respectively covered on the plurality of predicted air-pollution sets, wherein the plurality of coverage distribution proportions are used as the plurality of first predicted influencing results, and the IoT cloud platform is further configured to generate the feedback instruction when at least one of the plurality of coverage distribution proportions is greater than a distribution threshold, wherein the feedback instruction is used to control the smoke exhaust flue to reduce the gas emission.

7. The air pollution forecast management system of claim 3, wherein the IoT cloud platform is configured to respectively compute at least one coverage distribution between the first predicted exhaust gas set and each of the plurality of predicted air-pollution sets and to determine whether a pollution concentration among the at least one coverage distribution is greater than a concentration threshold, and the IoT cloud platform is further configured to generate the feedback instruction when the pollution concentration is greater than the concentration threshold, wherein the feedback instruction is used to control the smoke exhaust flue to decrease the gas emission.

8. The air pollution forecast management system of claim 5, wherein the IoT cloud platform is configured to perform a grid computation by using the currently-observed meteorological data and the predicted meteorological data, wherein the currently-observed meteorological data and the predicted meteorological data respectively comprise a first quantity of geographic location coordinates, and the IoT cloud platform is further configured to respectively expand the currently-observed meteorological data and the predicted meteorological data from the first quantity to a second quantity to obtain currently-observed meteorological grid data and predicted meteorological grid data, wherein the currently-observed meteorological grid data and the predicted meteorological grid data respectively comprise the second quantity of geographic location coordinates and meteorological grid data.

9. The air pollution forecast management system of claim 8, wherein the IoT cloud platform is configured to use, at the second time, the currently-observed meteorological grid data to compute a first grid exhaust gas set of the gas drifting from the first time to the second time, use the predicted meteorological grid data to compute a second grid exhaust gas set of the gas drifting from the second time to the third time, obtain a grid gas track of the gas drifting from the first time to the third time according to the exhaust gas set, the first grid exhaust gas set, and the second grid exhaust gas set, and tag the grid gas track on a map, wherein a resolution of the grid gas track is greater than the resolution of the predicted gas track on the map.

10. An air pollution forecast management method, comprising:
controlling gas exhausted from a smoke exhaust flue;
computing, at a second time after a first time, an exhaust gas set of the gas drifting from the first time to the second time by using currently-observed meteorological data;
receiving, at the second time, a plurality of air-pollution sets at a plurality of geographic locations;
in computing a plurality of influencing results of the plurality of air-pollution sets respectively associated with the exhaust gas set; and
generating a feedback instruction according to at least one of the pluralities of influencing results to control gas emission of the smoke exhaust flue.

11. The air pollution forecast management method of claim 10, further comprising:
using, at the second time, predicted meteorological data of a third time to compute a first predicted exhaust gas set of the exhaust gas set drifting from the second time to the third time;
using, at the second time, the predicted meteorological data of the third time to compute a plurality of first predicted air-pollution sets that the plurality of air-pollution sets locate at the third time;
computing a plurality of first predicted influencing results of the plurality of first predicted air-pollution sets respectively associated with the first predicted exhaust gas set; and
determining whether to generate the feedback instruction according to at least one of the pluralities of first predicted influencing results.

12. The air pollution forecast management method of claim 11, further comprising:
using, at the second time, the predicted meteorological data of a fourth time to compute a second predicted exhaust gas set that the first predicted exhaust gas set drift from the third time to the fourth time and computing a plurality of second predicted air-pollution sets that the plurality of first predicted air-pollution sets locate at the fourth time; and
computing a plurality of second predicted influencing results of the plurality of second predicted air-pollution sets respectively associated with second predicted exhaust gas set to determine whether to generate the feedback instruction at the second time according to at least one of the pluralities of second predicted influencing results.

13. The air pollution forecast management method of claim 12, further comprising:
obtaining a predicted gas track of the gas drifting from the second time to the fourth time according to the exhaust gas set, the first predicted exhaust gas set, and the second predicted exhaust gas set; and
tagging the predicted gas track on a map.

14. The air pollution forecast management method of claim 11, wherein determining whether to generate the feedback instruction according to at least one of the pluralities of first predicted influencing results comprises:
computing a plurality of coverage distribution proportions of the first predicted exhaust gas set respectively covered on the plurality of predicted air-pollution sets, wherein the plurality of coverage distribution proportions is used as the plurality of first predicted influencing results; and
generating the feedback instruction when at least one of the pluralities of coverage distribution proportions is greater than a distribution threshold, wherein the feedback instruction is used to control the smoke exhaust flue to reduce the gas emission.

15. The air pollution forecast management method of claim 11, wherein determining whether to generate the feedback instruction according to at least one of the pluralities of first predicted influencing results comprises:
computing at least one coverage distribution respectively between the first predicted exhaust gas set and each of the plurality of predicted air-pollution sets;
determining whether a pollution concentration among the at least one coverage distribution is greater than a concentration threshold; and
generating the feedback instruction when the pollution concentration is greater than the concentration threshold, wherein the feedback instruction is used to control the smoke exhaust flue to decrease the gas emission.

16. The air pollution forecast management method of claim 11, further comprising:
receiving a plurality of first sensing data through a plurality of air quality management devices;
receiving a plurality of second sensing data through a plurality of local air-quality monitoring stations;
receiving a plurality of to-be-calibrated sensing data through a plurality of micro air-quality monitoring stations; and
executing a predictor-corrector model by an IoT cloud platform to predict and correct the to-be-calibrated sensing data by referring to the first sensing data and the second sensing data to generate a plurality of calibrated measurement values of the plurality of micro air-quality monitoring stations.

17. The air pollution forecast management method of claim 16, wherein the predictor-corrector model comprises a machine learning model, a regression analysis model, an outliner analysis model, a median computation, an average computation, or a normal distribution computation.
